(19) United States
(12) Patent Application Publication
(10) Pub. No.: US 2003/0229303 A1
(43) Pub. Date: Dec. 11, 2003

(54) Expandable glaucoma implant and methods of use

(76) Inventors: David S. Haffner, Mission Viejo, CA (US); Gregory T. Smedley, Aliso Viejo, CA (US); Thomas W. Burns, Dana Point, CA (US); Robert D. Anello, Mission Viejo, CA (US); Hosheng Tu, Newport Coast, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/395,646

(22) Filed: Mar. 21, 2003

Related U.S. Application Data
(60) Provisional application No. 60/366,968, filed on Mar. 22, 2002.
(60) Provisional application No. 60/445,893, filed on Feb. 7, 2003.

(51) Int. Cl. A61F 2/14

(52) U.S. Cl. 604/8

(57) ABSTRACT

Disclosed is an implant for use in an eye with glaucoma, the implant including an inlet section in fluid communication with an outlet section, the inlet section being sized and shaped to fit at least partially in the anterior chamber of the eye, and the outlet section being sized and shaped to fit at least partially in Schlemm's canal of the eye. The implant also includes an expandable substrate suitable for expansion in the eye to assist in retaining the implant in the eye.

### CROSS REFERENCE TO RELATED APPLICATIONS

This application claims the priority benefit of U.S. Provisional Application No. 60/366,968, entitled “Expandable Stent and Methods Thereof for Glaucoma Treatment ab Interno,” filed Mar. 22, 2002, and U.S. Provisional Application No. 60/445,893, entitled “Hydrogel Loaded Implant and Methods of Use,” filed Feb. 7, 2003, the entireties of which are hereby incorporated by reference.

### BACKGROUND OF THE INVENTION

1. Field of the Invention

This invention relates to reducing intraocular pressure within the animal eye. More particularly, this invention relates to a treatment of glaucoma wherein aqueous humor is permitted to flow out of the anterior chamber of the eye through a surgically implanted pathway. Furthermore, this invention relates to expanding a distal portion of the stent once that portion is placed within a target body channel.

2. Description of the Related Art

As is well known in the art, a human eye is a specialized sensory organ capable of light reception and is able to receive visual images. Aqueous humor is a transparent liquid that fills the region between the cornea, at the front of the eye, and the lens. A trabecular meshwork, located in the anterior chamber angle formed between the iris and the cornea, serves as a drainage channel for aqueous humor from the anterior chamber, which maintains a balanced pressure within the anterior chamber of the eye.

About two percent of people in the United States have glaucoma. Glaucoma is a group of eye diseases encompassing a broad spectrum of clinical presentations, etiologies, and treatment modalities. Glaucoma causes pathological changes in the optic nerve, visible on the optic disk, and it causes corresponding visual field loss, resulting in blindness if untreated. Lowering intraocular pressure is the major treatment goal in all glaucomas.

In glaucomas associated with an elevation in eye pressure (intraocular hypertension), the source of resistance to outflow is mainly in the trabecular meshwork. The tissue of the trabecular meshwork allows the aqueous humor (hereinafter referred to as “aqueous”) to enter Schlemm's canal, which then empties into aqueous collector channels in the posterior wall of Schlemm's canal and then into aqueous veins, which form the episcleral venous system. Aqueous is continuously secreted by a ciliary body around the lens, so there is a constant flow of aqueous from the ciliary body to the anterior chamber of the eye. Pressure within the eye is determined by a balance between the production of aqueous and its exit through the trabecular meshwork (major route) and uveal scleral outflow (minor route). The portion of the trabecular meshwork adjacent to Schlemm's canal (the juxtacanilicular meshwork) causes most of the resistance to aqueous outflow.

Glaucoma is broadly classified into two categories: closed-angle glaucoma, also known as angle closure glaucoma, and open-angle glaucoma. Closed-angle glaucoma is caused by closure of the anterior chamber angle by contact between the iris and the inner surface of the trabecular meshwork. Closure of this anatomical angle prevents normal drainage of aqueous from the anterior chamber of the eye. Open-angle glaucoma is any glaucoma in which the exit of aqueous through the trabecular meshwork is diminished while the angle of the anterior chamber remains open. For most cases of open-angle glaucoma, the exact cause of diminished filtration is unknown. Primary open-angle glaucoma is the most common of the glaucomas, and is often asymptomatic in the early to moderately advanced stages of glaucoma. Patients may suffer substantial, irreversible vision loss prior to diagnosis and treatment. However, there are secondary open-angle glaucomas that may include edema or swelling of the trabecular spaces (e.g., from corticosteroid use), abnormal pigment dispersion, or diseases such as hyperthyroidism that produce vascular congestion.

All current therapies for glaucoma are directed toward decreasing intraocular pressure. Currently recognized categories of drug therapy for glaucoma include: (1) Miotics (e.g., pilocarpine, carbachol, and acetylcholinesterase inhibitors), (2) Sympathomimetics (e.g., epinephrine and dipivalylepinephxine), (3) Beta-blockers (e.g., betaxolol, levobunolol and timolol), (4) Carbonic anhydrase inhibitors (e.g., acetazolamide, methazolamide and ethoxzolamide), and (5) Prostaglandins (e.g., metabolite derivatives of arachindonic acid). Medical therapy includes topical ophthalmic drops or oral medications that reduce the production of aqueous or increase the outflow of aqueous. However, drug therapies for glaucoma are sometimes associated with significant side effects. The most frequent and perhaps most serious drawback to drug therapy is that patients, especially the elderly, often fail to correctly self-medicate. Such patients forget to take their medication at the appropriate times or else administer eye drops improperly, resulting in under- or over-dosing. Because the effects of glaucoma are irreversible, when patients dose improperly, allowing ocular concentrations to drop below appropriate therapeutic levels, further permanent damage to vision occurs. Furthermore, current drug therapies are targeted to be deposited directly into the ciliary body where the aqueous is produced. And, current therapies do not provide for a continuous slow-release of the drug. When drug therapy fails, surgical therapy is pursued.

Surgical therapy for open-angle glaucoma consists of laser trabeculoplasty, trabeculectomy, and implantation of aqueous shunts after failure of trabeculectomy or if trabeculectomy is unlikely to succeed. Trabeculectomy is a major surgery that is widely used and is augmented with topically applied anticancer drugs, such as 5-flurouracil or mitomycin-C to decrease scarring and increase the likelihood of surgical success.

Approximately 100,000 trabeculectomies are performed on Medicare-age patients per year in the United States. This number would likely increase if ocular morbidity associated with trabeculectomy could be decreased. The current morbidity associated with trabeculectomy consists of failure (10-15%); infection (a life long risk of 2-5%); choroidal hemorrhage, a severe internal hemorrhage from low intraocular pressure, resulting in visual loss (1%); cataract formation; and hypotony maculopathy (potentially reversible visual loss from low intraocular pressure). For these reasons, surgeons have tried for decades to develop a workable surgery for the trabecular meshwork.

The surgical techniques that have been tried and practiced are goniotomy/trabeculotomy and other mechanical disruptions of the trabecular meshwork, such as trabeculopuncture, goniophotoablation; laser trabecular ablation, and goniocurretage. These are all major operations and are briefly described below.

Goniotomy and trabeculotomy are simple and directed techniques of microsurgical dissection with mechanical disruption of the trabecular meshwork. These initially had early favorable responses in the treatment of open-angle glaucoma. However, long-term review of surgical results showed only limited success in adults. In retrospect, these procedures probably failed due to cellular repair and fibrosis mechanisms and a process of “filling in.” Filling in is a detrimental effect of collapsing and closing in of the created opening in the trabecular meshwork. Once the created openings close, the pressure builds back up and the surgery fails.

Q-switched Neodynium (Nd) YAG lasers also have been investigated as an optically invasive trabeculopuncture technique for creating full-thickness holes in trabecular meshwork. However, the relatively small hole created by this trabeculopuncture technique exhibits a filling-in effect and fails.

Goniophotoablation is disclosed by Berlin in U.S. Pat. No. 4,846,172 and involves the use of an excimer laser to treat glaucoma by ablating the trabecular meshwork. This method did not succeed in a clinical trial. Hill et al. used an Erbium YAG laser to create full-thickness holes through trabecular meshwork (Hill et al., Lasers in Surgery and Medicine 11:341346, 1991). This laser trabecular ablation technique was investigated in a primate model and a limited human clinical trial at the University of California, Irvine. Although ocular morbidity was zero in both trials, success rates did not warrant further human trials. Failure was again from filling in of surgically created defects in the trabecular meshwork by repair mechanisms. Neither of these is a viable surgical technique for the treatment of glaucoma.

Goniocurretage is an “ab interno” (from the inside), mechanically disruptive technique that uses an instrument similar to a cyclodialysis spatula with a microcurrette at the tip. Initial results were similar to trabeculotomy: it failed due to repair mechanisms and a process of filling in.

Although trabeculectomy is the most commonly performed filtering surgery, viscocanulostomy (VC) and nonpenetrating trabeculectomy (NPT) are two new variations of filtering surgery. These are “ab externo” (from the outside), major ocular procedures in which Schlemm's canal is surgically exposed by making a large and very deep scleral flap. In the VC procedure, Schlemm's canal is cannulated and viscoelastic substance injected (which dilates Schlemm's canal and the aqueous collector channels). In the NPT procedure, the inner wall of Schlemm's canal is stripped off after surgically exposing the canal.

Trabeculectomy, VC, and NPT involve the formation of an opening or hole under the conjunctiva and scleral flap into the anterior chamber, such that aqueous is drained onto the surface of the eye or into the tissues located within the lateral wall of the eye. These surgical operations are major procedures with significant ocular morbidity. When trabeculectomy, VC, and NPT are thought to have a low chance for success, a number of implantable drainage devices have been used to ensure that the desired filtration and outflow of aqueous through the surgical opening will continue. The risk of placing a glaucoma drainage device also includes hemorrhage, infection, and diplopia (double vision).

Examples of implantable shunts and surgical methods for maintaining an opening for the release of aqueous from the anterior chamber of the eye to the sclera or space beneath the conjunctiva have been disclosed in, for example, Hsia et al., U.S. Pat. No. 6,059,772 and Baerveldt, U.S. Pat. No. 6,050,970.

All of the above embodiments and variations thereof have numerous disadvantages and moderate success rates. They involve substantial trauma to the eye and require great surgical skill in creating a hole through the full thickness of the sclera into the subconjunctival space. The procedures are generally performed in an operating room and involve a prolonged recovery time for vision. The complications of existing filtration surgery have prompted ophthalmic surgeons to find other approaches to lowering intraocular pressure.

Because the trabecular meshwork and juxtacanilicular tissue together provide the majority of resistance to the outflow of aqueous, they are logical targets for surgical removal in the treatment of open-angle glaucoma. In addition, minimal amounts of tissue need be altered and existing physiologic outflow pathways can be utilized.

As reported in Arch. Ophthalm. (2000) 118:412, glaucoma remains a leading cause of blindness, and filtration surgery remains an effective, important option in controlling glaucoma. However, modifying existing filtering surgery techniques in any profound way to increase their effectiveness appears to have reached a dead end. The article further states that the time has come to search for new surgical approaches ihat may provide better and safer care for patients with glaucoma.

What is needed, therefore, is a site-specific treatment method for placing a trabecular microstent into the eye for diverting aqueous humor from the anterior chamber into Schlemm's canal. In some aspects of the present invention, a trabecular microstent is provided with at least a portion sized and configured to expand after implantation that is adapted suitably for retention within Schlemm's canal or other body opening.

### SUMMARY OF THE INVENTION

A device and methods are provided for improved treatment of elevated intraocular pressure due to glaucoma. A hollow trabecular microstent is adapted for implantation within a trabecular meshwork of an eye such that aqueous humor flows controllably from the anterior chamber of the eye to Schlemm's canal, bypassing the trabecular meshwork. In one embodiment, the trabecular microstent comprises a quantity of a therapeutic agent effective in treating glaucoma, which is controllably released from the device into tissue of the trabecular meshwork and/or Schlemm's canal. Depending upon the specific treatment contemplated, therapeutic agents may be utilized in conjunction with the trabecular microstent such that aqueous flow either increases or decreases as desired. Placement of the trabecular microstent within the eye and incorporation, and eventual release, of a proven therapeutic glaucoma therapy can inhibit or slow the effects of glaucoma.

In one aspect of the present invention, a trabecular microstent is provided that is implantable within an eye, the microstent comprising an inlet section having an inlet opening and an inlet circumferential periphery; an outlet section having an outlet opening and an outlet circumferential periphery; a middle section having a middle lumen and a middle circumferential periphery. The middle section is attached to the outlet and inlet sections, the middle lumen being in fluid communication with both the outlet opening and the inlet opening, wherein a swellable substrate is coated about at least a portion of the outer circumferential periphery of the outlet section, and wherein the substrate swellably expands radially outwardly after implantation that is adapted suitably for retention within the eye.

In another aspect of the present invention, a microstent is provided that is implantable within a body channel comprising a tubular mesh having an outer circumferential periphery; and a swellable substrate incorporated about at least a portion of the outer circumferential periphery of the tubular mesh, wherein the substrate swellably expands radially outwardly after implantation that is adapted suitably for retention within the body channel.

In still another aspect of the present invention, a method of implanting a swellable microstent within an eye is provided, comprising creating an incision through a conjunctival tissue at a limbus; radially incising an junction between an angle tissue and sclera, which is surgically extended until Schlemm's canal is entered posteriorly; and placing the swellable microstent between the anterior chamber and Schlemm's canal of the eye, wherein the microstent swells after implantation that is adapted suitably for retention within the eye.

In some aspects of the present invention, a method of implanting a swellable microstent within an eye is provided, comprising creating an incision through a cornea; incising an opening through trabecular meshwork, which is surgically extended until Schlemm's canal is entered anteriorly; and placing the swellable microstent between the anterior chamber and Schlemm's canal of the eye, wherein the microstent swells after implantation that is adapted suitably for retention within the eye.

One aspect of the invention includes an implant that is implantable in an eye with glaucoma, the implant comprising an inlet section in fluid communication with an outlet section, the inlet section being sized and shaped to fit at least partially in the anterior chamber of the eye, and the outlet section being sized and shaped to fit at least partially in Schlemm's canal of the eye; wherein the implant comprises an expandable substrate suitable for expansion in the eye to assist in retaining the implant in the eye.

In some embodiments the expandable substrate is at (including in or on) the inlet section, the outlet section, or both.

In some embodiments the implant comprises a material selected from the group consisting of titanium, stainless steel, silicone, polyurethane, polyvinyl alcohol, polyvinyl pyrolidone, collagen, heparinized collagen, polytetrafluoroethylene, expanded polytetrafluoroethylene, fluorinated polymer, fluorinated elastomer, flexible fused silica, polyolefin, polyester, and polysilicon.

In some embodiments the implant comprises a biodegradable material selected from the group consisting of poly(lactic acid), polyethylene-vinyl acetate, poly(lactic-co-glycolic acid), poly(D,L-lactide), poly(D,L-lactide-co-trimethylene carbonate), poly(caprolactone), and poly(glycolic acid).

In some embodiments the expandable substrate is a hydrogel. In further embodiments the hydrogel is hydrolytically degradable.

In some embodiments the implant further comprises at least one therapeutic agent selected from the group consisting of heparin, beta-adrenergic antagonists, TGF-beta, anti-glaucoma drugs, and antibiotics.

In some embodiments the implant further comprises at least one therapeutic agent selected from the group consisting of a gene, a growth factor, and an enzyme.

In some embodiments the implant is substantially axisymmetric.

One aspect of the invention includes a surgical method treating glaucoma in an eye comprising incising through the sclera of the eye and into Schlemm's canal of the eye; placing an implant, having an inlet section in fluid communication with an outlet section, through the scleral incision into the eye such that the inlet section of the implant resides at least partially in the anterior chamber of the eye, and the outlet section resides at least partially in Schlemm's canal of the eye; and expanding a substrate on the implant to assist in retaining the implant in the eye. “Expanding a substrate” may be active or passive, and thus includes allowing the substrate, such as a hydrogel, to expand by itself based on its own inherent properties.

One aspect of the invention includes a surgical method treating glaucoma in an eye comprising incising through the cornea of the eye; placing an implant, having an inlet section in fluid communication with an outlet section, through the corneal incision into the anterior chamber of the eye; positioning the implant such that the inlet section of the implant resides at least partially in the anterior chamber of the eye, and the outlet section resides at least partially in Schlemm's canal of the eye; and expanding a substrate on the implant to assist in retaining the implant in the eye.

### BRIEF DESCRIPTION OF THE DRAWINGS

FIG. 1 is a coronal, cross-sectional view of an eye.

FIG. 2 is an enlarged cross-sectional view of the anterior chamber angle of the eye of FIG. 1.

FIG. 3 is an elevation view of one embodiment of a trabecular microstent.

FIG. 4A is a first embodiment of the front cross-sectional view of the middle section of the axisymmetric trabecular stenting device of FIG. 3 loaded with hydrogel before hydrogel swelling.

FIG. 4B is the front cross-sectional view of the middle section of the axisymmetric trabecular stenting device of FIG. 4A after hydrogel swelling.

FIG. 5A is a second embodiment of the front cross-sectional view of the middle section of the axisymmetric trabecular stenting device of FIG. 3 loaded with hydrogel before hydrogel swelling.

FIG. 5B is the front cross-sectional view of the middle section of the axisymmetric trabecular stenting device of FIG. 5A after hydrogel swelling.

FIG. 6 is a side elevational view of another preferred embodiment of a trabecular microstent.

FIG. 7 illustrates the trabecular microstent of FIG. 6 at an initial deployed state.

FIG. 8 illustrates the trabecular microstent of FIG. 6 at a later-stage deployed state.

FIG. 9 is an enlarged, cross-sectional view of a preferred method of implanting a trabecular stenting device within an eye.

FIG. 10 is a cross-sectional view of a microstent with an expandable basket.

### DETAILED DESCRIPTION OF EXEMPLARY EMBODIMENTS

Some exemplary embodiments of the invention described below relate particularly to surgical and therapeutic treatment of glaucoma through reduction of intraocular pressure. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described below.

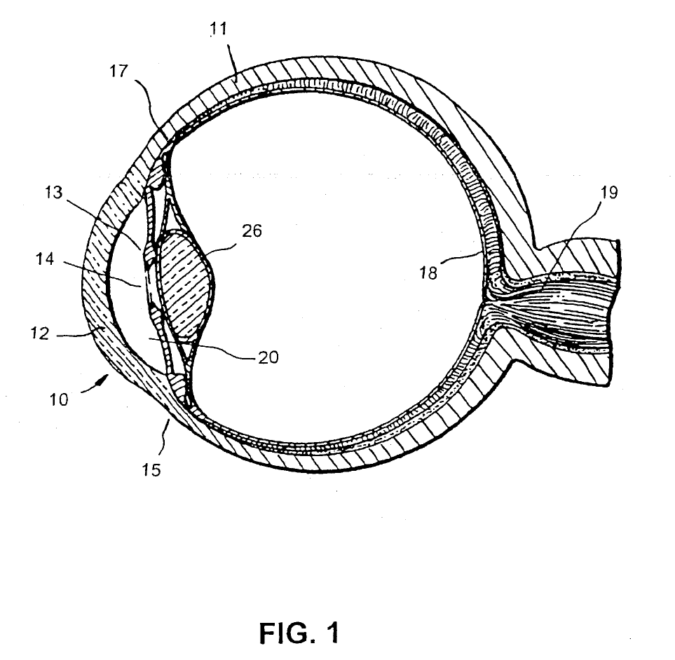

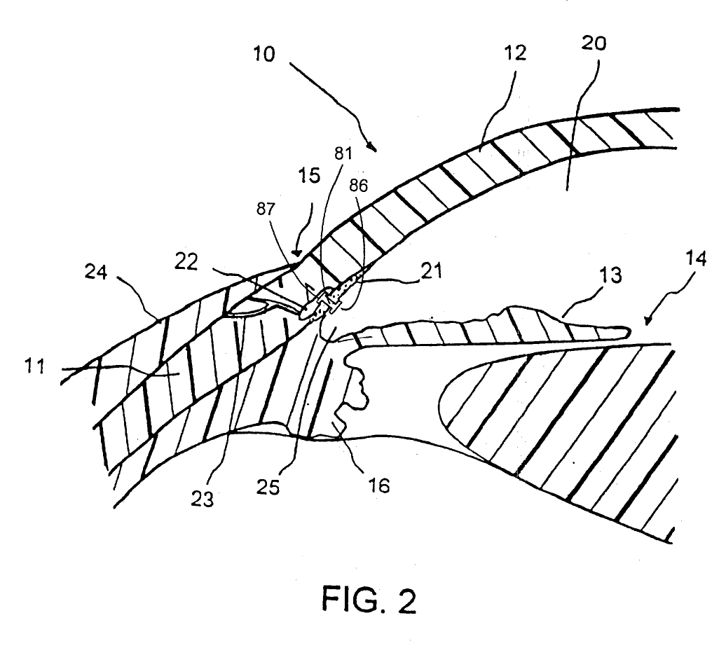

FIG. 1 is a cross-sectional view of an eye 10, while FIG. 2 is a close-up view showing the relative anatomical locations of a trabecular meshwork 21, the anterior chamber 20, and a Schlemm's canal 22. A sclera 11 is a thick collagenous tissue that covers the entire eye 10 except a portion that is covered by a cornea 12. The cornea 12 is a thin transparent tissue that focuses and transmits light into the eye and through a pupil 14, which is a circular hole in the center of an iris 13 (colored portion of the eye). The cornea 12 merges into the sclera 11 at a juncture referred to as a limbus 15. A ciliary body 16 extends along the interior of the sclera 11 and is coextensive with a choroid 17. The choroid 17 is a vascular layer of the eye 10, located between the sclera 11 and a retina 18. An optic nerve 19 transmits visual information to the brain and is the anatomic structure that is progressively destroyed by glaucoma.

The anterior chamber 20 of the eye 10, which is bound anteriorly by the cornea 12 and posteriorly by the iris 13 and a lens 26, is filled with aqueous humor (hereinafter referred to as “aqueous”). Aqueous is produced primarily by the ciliary body 16, then moves anteriorly through the pupil 14 and reaches the anterior chamber angle 25, formed between the iris 13 and the cornea 12. In a normal eye, aqueous is removed from the anterior chamber 20 through the trabecular meshwork 21. Aqueous passes through the trabecular meshwork 21 into Schlemm's canal 22 and thereafter through a plurality of aqueous veins 23, which merge with blood-carrying veins, and into systemic venous circulation. Intraocular pressure is maintained by an intricate balance between secretion and outflow of aqueous in the manner described above. Glaucoma is, in most cases, characterized by an excessive buildup of aqueous in the anterior chamber 20, which leads to an increase in intraocular pressure. Fluids are relatively incompressible, and thus intraocular pressure is distributed relatively uniformly throughout the eye 10.

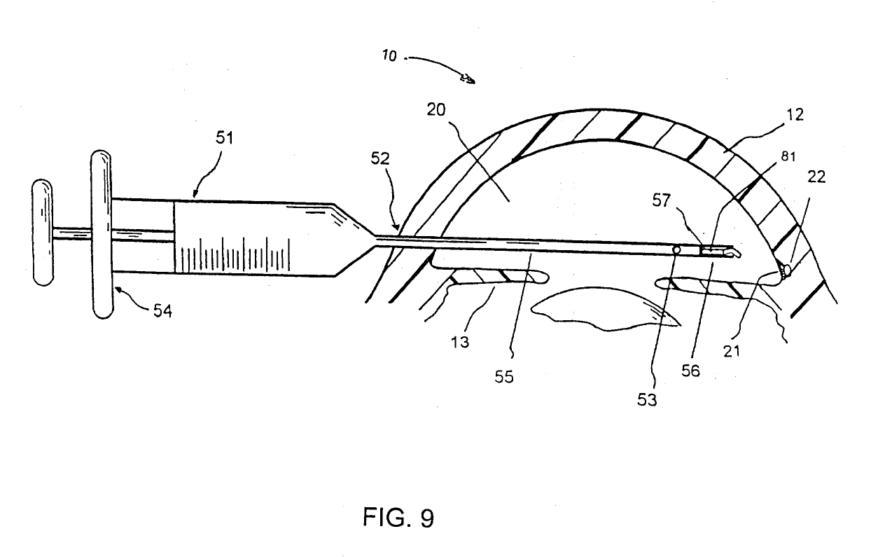

As shown in FIG. 2, the trabecular meshwork 21 is adjacent to a small portion of the sclera 11. Exterior to the sclera 11 is a conjunctiva 24. Traditional procedures that create a hole or opening for implanting a device through the tissues of the conjunctiva 24 and sclera 11 involve extensive surgery, as compared to surgery for implanting a device, as described herein, which ultimately resides entirely within the confines of the sclera 11 and cornea 12. A microstent 81 is shown placed through trabecular meshwork 21 having a distal opening 83 exposed to Schlemm's canal 22 and a proximal opening 86 exposed to the anterior chamber 20 of the eye 10. FIG. 9 generally illustrates the use of one embodiment of a trabecular microstent 81 for establishing an outflow pathway, passing through the trabecular meshwork 21, which is discussed in greater detail below.

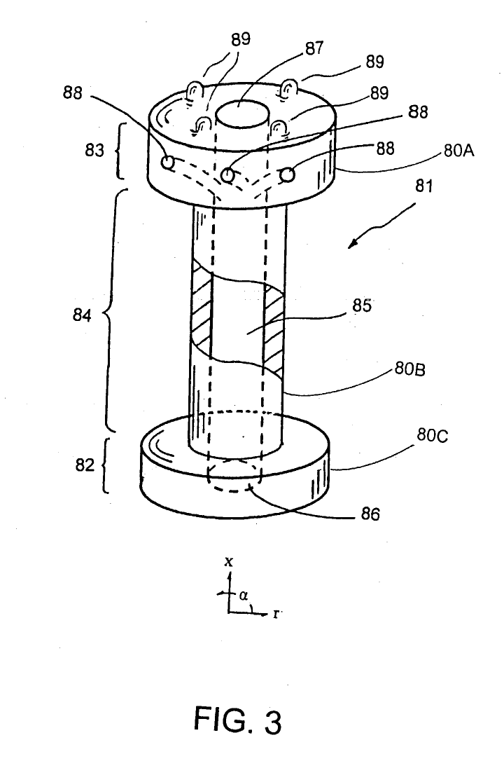

FIG. 3 illustrates a preferred embodiment of a hollow trabecular microstent 81, which facilitates the outflow of aqueous from the anterior chamber 20 into Schlemm's canal 22, and subsequently into the aqueous collectors and the aqueous veins so that intraocular pressure is reduced. In the illustrated embodiment, the trabecular microstent 81 comprises an inlet section 82, having an inlet (proximal) opening 86, a middle section 84, and an outlet section 83 having at least one (distal) opening 87, 88. The middle section 84 may be an extension of, or may be coextensive with, the inlet section 82. The device 81 comprises at least one lumen 85 within section 84, which is in fluid communication with the inlet opening 86 and the outlet opening 87, 88, thereby facilitating transfer of aqueous through the device 81. In one preferred embodiment, the outlet side openings 88, each of which is in fluid communication with the lumen 85 for transmission of aqueous, are arranged spaced apart around the outlet circumferential periphery 80A of the outlet section 83. In another aspect, the outlet openings 88 are located and configured to enable jet-like infusing fluid impinging any specific region of Schlemm's canal tissue suitably for tissue stimulation.

As will be apparent to a person skilled in the art, the lumen 85 and the remaining body of the outlet section 83 may have a cross-sectional shape that is oval, circular, or other appropriate shape. Preferably, the middle section 84 has a length that is roughly equal to a thickness of the trabecular meshwork 21, which typically ranges between about 100 μm and about 300 μm. As shown in FIG. 3, the trabecular microstent may be an axisymmetric one or a circumferentially axisymmetric one with respect to a straight axial line of the microstent 81. An axisymmetric device 81 has a coordinate of x, r and angle α as shown in FIG. 3, rather than depending on a conventional coordinate of x, y, and z.

To further stent Schlemm's canal after implanting the axisymmetric device 81, a plurality of elevated (that is, protruding axially) supporting posts, legs, pillars, or stenting standoffs 89 is located at the distal-most end of the outlet section 83 sized and configured for allowing media (for example, aqueous, liquid, balanced salt solution, viscoelastic fluid, therapeutic agents, or the like) to be transported freely.

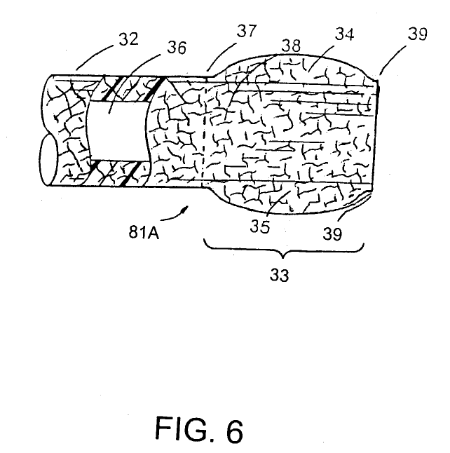

The trabecular microstent 81 may be made by molding, thermo-forming, or other micro-machining techniques. The trabecular microstent 81 (and 81A in FIG. 6) preferably comprises a biocompatible material such that inflammation arising due to irritation between the outer surface of the device and the surrounding tissue is minimized. Biocompatible materials which may be used for the device 81, 81A preferably include, but are not limited to, titanium, stainless steel, medical grade silicone, e.g., Silastic™, available from Dow Coming Corporation of Midland, Mich.; and polyurethane, e.g., Pellethane™, also available from Dow Coming Corporation. In other embodiments, the device may comprise other types of biocompatible material, such as, by way of example, polyvinyl alcohol, polyvinyl pyrolidone, collagen, heparinized collagen, polytetrafluoroethylene, expanded polytetrafluoroethylene, fluorinated polymer, fluorinated elastomer, flexible fused silica, polyolefin, polyester, polysilicon, and/or a mixture of the aforementioned biocompatible materials, and the like. In another aspect, the microstent is made of a biodegradable material selected from a group consisting of poly(lactic acid), polyethylene-vinyl acetate, poly(lactic-co-glycolic acid), poly(D,L-lactide), poly(D,L-lactide-co-trimethylene carbonate), poly(caprolactone), poly(glycolic acid), and copolymer thereof.

In still other embodiments, composite biocompatible material may be used, wherein a surface material may be used in addition to one or more of the aforementioned materials. For example, such a surface material may include polytetrafluoroethylene (PTFE) (such as Teflon™), polyimide, hydrogel, heparin, therapeutic drugs (such as beta-adrenergic antagonists, TGF-beta, and other anti-glaucoma drugs, or antibiotics), and the like.

In one embodiment, the device 81, 81A may be made of a biodegradable (also including bioerodible) material admixed with a substance for substance slow-release into ocular tissues. In another embodiment, polymer films (substrate) may function as substance containing release devices whereby the polymer films may be coupled or secured to the device 81, 81A. The polymer films may be designed to permit the controlled release of the substance at a chosen rate and for a selected duration, which may also be episodic or periodic. Such polymer films may be synthesized such that the substance is bound to the surface or resides within a pore in the film so that the substance is relatively protected from enzymatic attack. The polymer films may also be modified to alter their hydrophilicity, hydrophobicity and vulnerability to platelet adhesion and enzymatic attack. In one embodiment, the polymer film is a swellable substrate, such as hydrogel.

The device or microstent 81 may be used for a direct release of pharmaceutical preparations into ocular tissues. As discussed above, the pharmaceuticals may be compounded within the device 81, 81A, form a coating on the device, or mixed with the hydrogel followed by coating onto the outer periphery of the device. Any known drug therapy for glaucoma may be utilized.

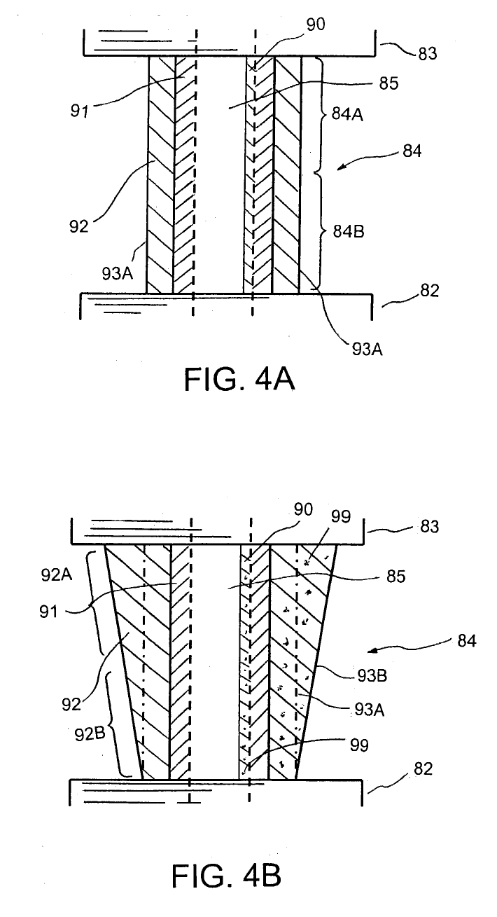

In one embodiment, FIG. 4A shows a front cross-sectional view of the middle section 84 of the axisymmetric trabecular stenting device 81 of FIG. 3 loaded with hydrogel before hydrogel swelling, while FIG. 4B shows the same front cross-sectional view of the middle section 84 of the axisymmetric trabecular stenting device of FIG. 4A after hydrogel swelling. In one aspect, the first hydrogel layer 90 is loaded or incorporated onto at least a portion of the lumen surface 85. In another aspect, a second hydrogel layer 92 is loaded onto the outer surface (that is, the circumferential periphery 80B) of the middle section 84. In some aspect, the distal middle section 84A has a relatively thicker hydrogel layer than the proximal middle section 84B so as to cause the distal middle section 84A to swell more (and become thicker in thickness) to hold the microstent firmly in place (by exerting force radially against the squeezed trabecular meshwork tissue 21) after implantation. Hydrogel typically contains more than 50% liquid-filled space (that is, there is a void when dehydrated). Trabecular meshwork tissue may tend to penetrate into the liquid-filled space/void as healing progresses. The hydrogel is compatible with the microstent material and construct, and is biocompatible with the ocular tissue.

As illustrated, the hydrogel layer 92 at the distal middle section 84A has higher swelling ratio (the swelling ratio is defined as the ratio of the final volume divided by the initial volume of the hydrogel) than the hydrogel at the proximal middle section 84B (FIGS. 4A before hydrogel swelling). This disproportional swelling causes the distal middle hydrogel layer 92A to swell more (and become thicker) than the proximal middle hydrogel layer 92B enabling the microstent 81 anchored firmly in place (FIG. 4B after hydrogel swelling). In one aspect, the non-swelling portion 91 of the stent middle section 84 has a constant thickness. The very outer surface of the middle section 84 is designated as 93A before swelling and as 93B after swelling. The swelling ratio of a hydrogel may be controlled by adding non-swelling polymer into a swellable hydrogel or by adding a second hydrogel with different swelling ratio into the first swellable hydrogel.

Accordingly, one aspect of the present invention includes providing a trabecular microstent that is implantable within an eye, the microstent comprising an inlet section 82 having an inlet opening 86 and an inlet circumferential periphery 80C; an outlet section 83 having an outlet opening 87 and an outlet circumferential periphery 80A; a middle section 84 having a middle lumen 85 and a middle circumferential periphery 80B, wherein the middle section 84 is attached to the outlet section 83 and the inlet section 82. The middle lumen 85 is configured in fluid communication with both the outlet opening 87 and the inlet opening 86, wherein a swellable substrate is coated about at least a portion of the outer circumferential periphery 80A of the outlet section 83, and wherein the substrate swellably expands radially outwardly (in a direction essentially perpendicular to an axial line) after implantation that is adapted suitably for microstent retention within the eye.

A further aspect of the invention includes coating at least a portion of the middle circumferential periphery 80B of the middle section 84 with the swellable substrate, and wherein the substrate swellably expands radially outwardly after implantation in a direction essentially perpendicular to an axial line.

In one aspect, at least some therapeutic substances 99 are loaded onto the exterior hydrogel layer 92 or into the interior hydrogel layer 90 of the middle section 84 of the stenting device 81 enabling releasing into the trabecular meshwork 21 or to Schlemm's canal 22 upon device implantation. At least one therapeutic agent is mixed with the swellable substrate on the trabecular microstent, wherein the at least one therapeutic agent is selected from a group consisting of heparin, beta-adrenergic antagonists, TGF-beta, anti-glaucoma drugs, antibiotics, pharmaceutical agents, genes, growth factors, enzymes, and mixture thereof.

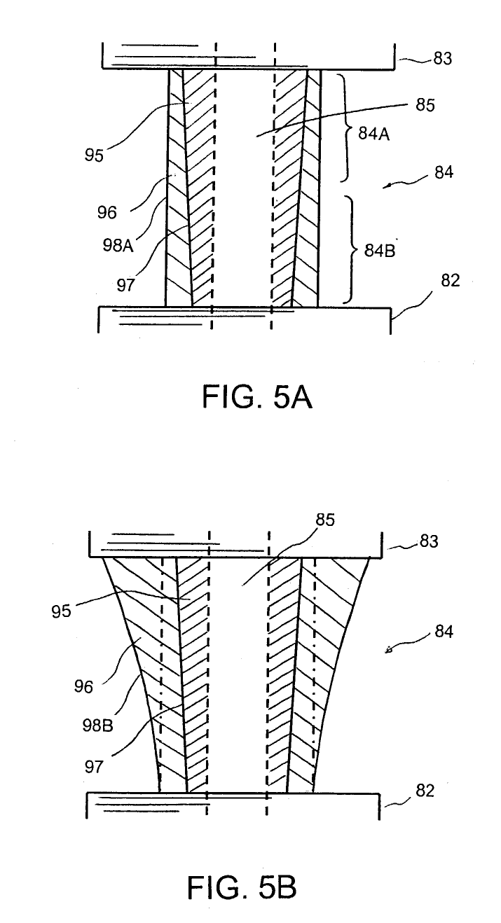

In a further aspect, the middle section 84 of the microstent 81 has a larger circumference at the distal middle section 84A than the circumference at the proximal middle section 84B. FIG. 5A shows a second embodiment of the front elevational cross-sectional view of the middle section 84 of the axisymmetric trabecular stenting device 81 of FIG. 3 loaded with hydrogel before hydrogel swelling, while FIG. 5B shows the front cross-sectional view of the middle section of the axisymmetric trabecular stenting device of FIG. 5A after hydrogel swelling. In one aspect, the hydrogel at the distal middle section 84A has a higher swelling ratio than the hydrogel at the proximal middle section 84B. After absorbing aqueous or liquid upon the microstent being implanted, the very outer surface of the middle section 84 is designated as 98A before swelling and as 98B after swelling. In some aspect, the wall thickness of the non-swelling portion 95 of the stent middle section 84 maintains constant before hydrogel swelling and after swelling.

As will be appreciated by those of ordinary skill in the art, the device 81 may advantageously be practiced with a variety of sizes and shapes without departing from the scope of the invention. Depending upon the distance between the anterior chamber 20 and the drainage vessel (e.g., a vein) contemplated, the devices 81 may have a length ranging from about 0.05 centimeters to over 1 centimeter. Preferably, the device 81 has an outside diameter ranging between about 30 μm and about 500 μm, with the lumen 85 having diameters ranging between about 20 μm and about 250 μm, respectively. In addition, the device 81 may have a plurality of lumens to facilitate transmission of multiple flows of aqueous or infusing fluid.

One preferred method for increasing aqueous outflow in the eye 10 of a patient, to reduce intraocular pressure therein, comprises bypassing the trabecular meshwork 21. In operation, the middle section 84 of the device 81 is advantageously placed across the trabecular meshwork 21 through a slit or opening. This opening can be created by use of a laser, a knife, thermal energy (radiofrequency, ultrasound, microwave), cryogenic energy, or other surgical cutting instrument ab interno or ab externo. The opening may advantageously be substantially horizontal, i.e., extending longitudinally in the same direction as the circumference of the limbus 15 (FIG. 2). Other opening directions may also be used, as well. The opening may advantageously be oriented at any angle, relative to the circumference of the limbus 15, that is appropriate for inserting the device 81 through the trabecular meshwork 21 and into Schlemm's canal 22 or other outflow pathway, as will be apparent to those skilled in the art. Furthermore, the outlet section 83 may be positioned into fluid collection channels of the natural outflow pathways. Such natural outflow pathways include Schlemm's canal 22, aqueous collector channels, aqueous veins, and episcleral veins.

The main purpose of the trabecular microstent is for transporting aqueous humor at the level of the trabecular meshwork and partially using existing the outflow pathway for aqueous humor, i.e., utilizing the entire outflow pathway except for the trabecular meshwork, which is bypassed by the trabecular microstent 81. In this manner, aqueous humor is transported into Schlemm's canal and subsequently into the aqueous collectors and the aqueous veins so that the intraocular pressure is properly maintained within a therapeutic range.

The copending patent application Ser. No. 09/549,350, filed Apr. 14, 2000, entire contents of which are incorporated herein by reference, discloses using a biocompatible material that hydrates and expands after implantation so that the microstent is locked into position around the trabecular meshwork opening or around the distal section of the microstent, while the material for the microstent may be selected from the group consisting of porous material, semi-rigid material, soft material, hydrophilic material, hydrophobic material, hydrogel, elastic material, and the like.

The copending patent application Ser. No. 09/847,523, filed May 2, 2001, entire contents of which are incorporated herein by reference, discloses a trabecular microstent having its surface coated with a coating material selected from one or more of the following: polytetrafluoroethylene (e.g., Teflon™), polyimide, hydrogel, heparin, hydrophilic compound, anti-angiogenic factor, anti-proliferative factor, therapeutic drugs, and the like.

The copending patent application Ser. No. 10/137,117, filed May 1, 2002, entire contents of which are incorporated herein by reference, discloses a microstent made of biocompatible porous material that imbibes aqueous humor. One or more materials for the device may be selected from the following material types: porous material, semi-rigid material, soft material, hydrophilic material, hydrophobic material, hydrogel, elastic material, biodegradable material, bioresorbable material, and the like. Further, the microstent material may be selected from the following: polyvinyl alcohol, polyvinyl pyrolidone, collagen, heparinized collagen, chemically treated collagen, polytetrafluoroethylene, expanded polytetrafluoroethylene, fluorinated polymer, fluorinated elastomer, flexible fused silica, silicone, polyurethane, poly(methyl methacrylate), acrylic, polyolefin, polyester, polysilicon, polypropylene, hydroxyapetite, titanium, gold, silver, platinum, biodegradable material, bioresorbable material, and mixture thereof. Furthermore, the trabecular microstent fabricated from a hydrogel material that expands with absorption of water. Desirably, this would enable the device to be inserted through a smaller incision in the trabecular meshwork. The subsequent expansion of the stent would advantageously enable it to latch in place in the trabecular meshwork.

The degradable poly(ethylene glycol) carbamate derivatives have potential applications in controlled hydrolytic degradation of hydrogels. In such degradable hydrogels, drugs may be either trapped in the gel and released by diffusion as the gel degrades, or they may be covalently bound through hydrolysable carbamate linkages. Hydrolysis of these carbamate linkages releases the amine drug at a controllable rate as the gel degrades. In some aspects of the invention, a trabecular microstent is provided that is loaded with drug-containing hydrogel for slow drug release. More particularly, the hydrogel can be hydrolytically degradable enabling drug release along with the rate of degradation.

In accordance with an embodiment, a hydrolytically degradable hydrogel is provided. The hydrogel comprises a backbone bonded to a crosslinking agent through a hydrolysable carbamate linkage. Typically, a suitable backbone can be any compound having an amino group, preferably at least two amino groups. Examples of such backbones include, but are not limited to, proteins, peptides, amino carbohydrates, aminolipids, poly(vinylamine), polylysine, poly(ethylene glycol) amines, pharmaceutical agents having an amino group, etc.

Gels are known materials that have mechanical properties that enable them to be stored without flowing significantly. Typically weaker gel materials can be loaded or incorporated onto a support, for example a trabecular microstent, a cardiovascular stent, or a peripheral vessel implant. Hydrogel materials can include a component in their composition that enables the materials to absorb water (including water-based liquids). It can absorb several times its own weight in water, resulting in significant swelling of the gel. It can be important for many applications for the ability of the gel material to absorb water to be balanced against loss of physical properties due to swelling when the water is absorbed.

A form of porous hydrogel materials can be provided by first creating gas pockets in the gel and then removing this gas. The removal of the gas creates a porous material, and the initial incorporation of sufficient gas allows one to create a material with an open, interconnected pore structure. Advantageous features of the resulting materials, in addition to their interconnected pore structure, may include that the pore structure is maintained over extended time periods and that the gels maintain a high mechanical integrity that allows cells penetration and proliferation without destruction or compression of the material. The approach is in contrast to other processing approaches typically used to achieve a porous structure with these types of materials (e.g., lyophilization) in which the porous nature is lost as the material rehydrates and/or the material is significantly weakened by the process.

The term “hydrogel” meant to broadly cover any biocompatible material that increases its volume after absorbing water, liquid or other suitable fluid.

In one embodiment, the trabecular microstent 81A (FIG. 6) comprises a hollow, elongate tubular element having an inlet section 32 and an outlet section 33 (also called distal section), wherein the outlet section 33 may comprise an expandable element that is adapted to be positioned inside Schlemm's canal for microstent stabilization. The outlet section 33 comprises a proximal interface 37 connected to the inlet section 32 and a distal end 39, wherein the swellable substrate comprises a core section 38 and edge sections 34, 35. The hollow elongate tubular element may comprise at least one lumen 36 for transporting aqueous from the anterior chamber 20 of an eye to Schlemm's canal 22. In one aspect, at least a portion of the outlet section 33 may be loaded with expandable, swellable substrate, such as hydrogel. In another aspect, at least a portion of the outlet section 33 may be made of a mesh material that is expandable. The “expandability” operation may be achieved by substrate swelling, mechanical forces and/or through the shape-memory property of a material.

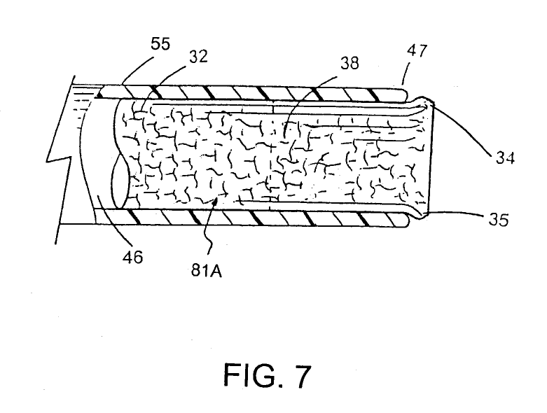

FIG. 7 shows the trabecular microstent of FIG. 6 in an initial stage of the deployment state. In one embodiment for stent delivery, the trabecular microstent 81A is placed inside a hollow delivery apparatus (also collectively called “an applicator”) 55. A delivery apparatus 55 comprises a distal end 47, wherein the outlet section 33 comprising a swellable or self-expandable substrate forms a substantially circular fashion when the trabecular microstent 81 is deployed out of the delivery apparatus 55. In one aspect, the edge section 34, 35 tends to swell and turn radially outwardly upon absorbing aqueous. The delivery apparatus 55 may comprise a deployment mechanism for deploying the trabecular microstent out of the distal end of the delivery apparatus. In one preferred embodiment, the distal end 47 of the applicator 55 may comprise a sharp edge for creating an opening or thinning the trabecular meshwork during the applicator delivery phase. In one embodiment, the deployment mechanism is a plunger. The delivery mechanism may be located at the handle of the delivery apparatus for deploying the trabecular microstent.

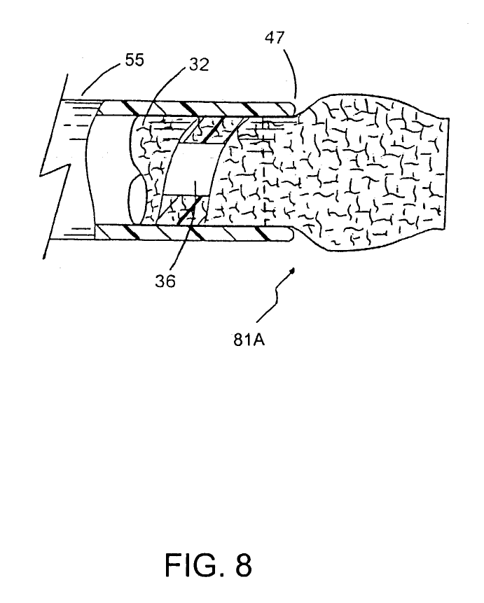

FIG. 8 the trabecular microstent of FIG. 6 at a later-stage deployment state. As the plunger continuously pushes ahead, and the distal end 47 of the delivery apparatus 55 retreats, the distal end section 33 of the microstent 81A continues to deploy in an expanded fashion. In one embodiment, this may be accomplished by pre-retracting or pre-compressing the microstent within the delivery apparatus before the delivery state. The outlet section of the trabecular microstent may be made of hydrogel or a material form selected from a group comprising porous form, semi-permeable form, and any form that is effective and appropriate to expand upon deployment.

In one embodiment, a method of placing a trabecular microstent into an opening through trabecular meshwork, the method comprises advancing and positioning a trabecular microstent having an expandable distal section 33 (in FIG. 6) through the opening. The trabecular microstent 81A may be an axisymmetric type or somewhat not so axisymmetric. In a further embodiment, a method of placing a trabecular microstent into an opening through diseased trabecular meshwork for transporting aqueous humor at the level of the trabecular meshwork and using an existing outflow pathway, the method comprises advancing and positioning a trabecular microstent out of the lumen of an applicator wherein the applicator (may also be known as a trephine) has cutting capability to create the opening.

As is well known in the art, a device coated or loaded with a slow-release substance can have prolonged effects on local tissue surrounding the device. The slow-release delivery can be designed such that an effective amount of substance is released over a desired duration. “Substance” or “therapeutic substance”, as used herein, is defined as any therapeutic or active drug that can stop, mitigate, slow-down or reverse undesired disease processes.

The device 81, 81A may be used for a direct release of pharmaceutical preparations into ocular tissues. As discussed above, the pharmaceuticals may be compounded within the device or form a coating (for example, swellable hydrogel) on the device 81, 81A. Any known drug therapy for glaucoma may be utilized, including but not limited to, the following:

U.S. Pat. No. 6,274,138, issued Aug. 14, 2001, and U.S. Pat. No. 6,231,853, issued May 15, 2001, the entire contents of both of which are incorporated herein by reference, disclose the function of mitochondria and toxic substances synthesized as a metabolic byproduct within mitochondria of cells. Perry and associates (Perry H D et al. “Topical cyclosporin A in the management of postkeratoplasty glaucoma” Cornea 16:284-288, 1997) report that topical cyclosporin-A has been shown to reduce post-surgical increases in intraocular pressure. It is proposed that such compounds with known effects on mitochondrial stability might be effective in treating trabecular meshwork. An antagonistic drug to neutralize the toxic byproduct or a stabilizing drug to effect mitochondrial stability is believed able to restore the mitochondria function and subsequently mitigate the dysfunction of the trabecular meshwork.

U.S. Pat. No. 6,201,001, issued Mar. 13, 2001, the entire contents of which are incorporated herein by reference, discloses Imidazole antiproliferative agents useful for neovascular glaucoma.

U.S. Pat. No. 6,228,873, issued May 8, 2001, the entire contents of which are incorporated herein by reference, discloses a new class of compounds that inhibit function of sodium chloride transport in the thick ascending limb of the loop of Henle, wherein the preferred compounds useful are furosemide, piretanide, benzmetanide, bumetanide, torasemide and derivatives thereof.

U.S. Pat. No. 6,194,415, issued Feb. 27, 2001, the entire contents of which are incorporated herein by reference, discloses a method of using quinoxalines (2-imidazolin-2-ylamino) in treating neural injuries (e.g. glaucomatous nerve damage).

U.S. Pat. No. 6,060,463, issued May 9, 2000, and U.S. Pat. No. 5,869,468, issued Feb. 9, 1999, the entire contents of which are incorporated herein by reference, disclose treatment of conditions of abnormally increased intraocular pressure by administration of phosphonylmethoxyalkyl nucleotide analogs and related nucleotide analogs.

U.S. Pat. No. 5,925,342, issued Jul. 20, 1999, the entire contents of which are incorporated herein by reference, discloses a method for reducing intraocular pressure by administration of potassium channel blockers.

U.S. Pat. No. 5,814,620, issued Sep. 29, 1998, the entire contents of which are incorporated herein by reference, discloses a method of reducing neovascularization and of treating various disorders associated with neovascularization. These methods include administering to a tissue or subject a synthetic oligonucleotide.

U.S. Pat. No. 5,767,079, issued Jun. 16, 1998, the entire contents of which are incorporated herein by reference, discloses a method for treatment of ophthalmic disorders by applying an effective amount of Transforming Growth Factor-Beta (TGF-beta) to the affected region.

U.S. Pat. No. 5,663,205, issued Sep. 2, 1997, the entire contents of which are incorporated herein by reference, discloses a pharmaceutical composition for use in glaucoma treatment which contains an active ingredient 5-[1-hydroxy-2-[2-(2-methoxyphenoxyl)ethylamino]ethyl]-2-methylbenzenesulfonamide. This agent is free from side effects, and stable and has an excellent intraocular pressure reducing activity at its low concentrations, thus being useful as a pharmaceutical composition for use in glaucoma treatment.

U.S. Pat. No. 5,652,236, issued Jul. 29, 1997, the entire contents of which are incorporated herein by reference, discloses pharmaceutical compositions and a method for treating glaucoma and/or ocular hypertension in the mammalian eye by administering thereto a pharmaceutical composition which contains as the active ingredient one or more compounds having guanylate cyclase inhibition activity. Examples of guanylate cyclase inhibitors utilized in the pharmaceutical composition and method of treatment are methylene blue, butylated hydroxyanisole and N-methylhydroxylamine.

U.S. Pat. No. 5,547,993, issued Aug. 20, 1996, the entire contents of which are incorporated herein by reference, discloses that 2-(4methylaminobutoxy) diphenylmethane or a hydrate or pharmaceutically acceptable salt thereof have been found useful for treating glaucoma.

U.S. Pat. No. 5,502,052, issued Mar. 26, 1996, the entire contents of which are incorporated herein by reference, discloses use of a combination of apraclonidine and timolol to control intraocular pressure. The compositions contain a combination of an alpha-2 agonist (e.g., para-amino clonidine) and a beta blocker (e.g., betaxolol).

U.S. Pat. No. 6,184,250, issued Feb. 6, 2001, the entire contents of which are incorporated herein by reference, discloses use of cloprostenol and fluprostenol analogues to treat glaucoma and ocular hypertension. The method comprises topically administering to an affected eye a composition comprising a therapeutically effective amount of a combination of a first compound selected from the group consisting of beta-blockers, carbonic anhydrase inhibitors, adrenergic agonists, and cholinergic agonists; together with a second compound.

U.S. Pat. No. 6,159,458, issued Dec. 12, 2000, the entire contents of which are incorporated herein by reference, discloses an ophthalmic composition that provides sustained release of a water soluble medicament formed by comprising a crosslinked carboxy-containing polymer, a medicament, a sugar and water.

U.S. Pat. No. 6,110,912, issued Aug. 29, 2000, the entire contents of which are incorporated herein by reference, discloses methods for the treatment of glaucoma by administering an ophthalmic preparation comprising an effective amount of a noncorneotoxic serine-threonine kinase inhibitor, thereby enhancing aqueous outflow in the eye and treatment of the glaucoma. In some embodiments, the method of administration is topical, whereas it is intracameral in other embodiments. In still further embodiments, the method of administration is intracanalicular.

U.S. Pat. No. 6,177,427, issued Jan. 23, 2001, the entire contents of which are incorporated herein by reference, discloses compositions of non-steroidal glucocorticoid antagonists for treating glaucoma or ocular hypertension.

U.S. Pat. No. 5,952,378, issued Sep. 14, 1999, the entire contents of which are incorporated herein by reference, discloses the use of prostaglandins for enhancing the delivery of drugs through the uveoscleral route to the optic nerve head for treatment of glaucoma or other diseases of the optic nerve as well as surrounding tissue. The method for enhancing the delivery to the optic nerve head comprises contacting a therapeutically effective amount of a composition containing one or more prostaglandins and one or more drug substances with the eye at certain intervals.

FIG. 9 generally illustrates a preferred method by which the trabecular microstent 81 is implanted within the eye 10. In the illustrated method, a delivery applicator 55 is provided, which preferably comprises a syringe portion 51 and a cannula portion 56, which contains at least one lumen 46 in fluid communication with the fluid supply 54. The cannula portion 56 preferably has a size of about 30 gauges. However, in other embodiments, the cannula portion 56 may have a size ranging between about 16 gauges and about 40 gauges. A holder at the distal portion of the cannula portion 56 for holding the device 81 may advantageously comprise a lumen, a sheath, a clamp, tongs, a space, and the like.

In the method illustrated in FIG. 9, the device 81 is placed into the lumen 46 of the delivery applicator 55 and then advanced to a desired implantation site within the eye 10. The delivery applicator 55 holds the device 81 securely during delivery and releases it when the practitioner initiates deployment actuator of the applicator 55.

In a preferred embodiment of trabecular meshwork surgery, a patient is placed in a supine position, prepped, draped, and appropriately anesthetized. A small incision 52 is then made through the cornea 12 with a self-trephining applicator 55. The incision 52 preferably has a surface length less than about 1.0 millimeter in length and may advantageously be self-sealing. Through the incision 52, the trabecular meshwork 21 is accessed, wherein an incision is made with a cutting means 47 enabling forming a hole on the trabecular meshwork 21 for stent placement. The hole on the trabecular meshwork can also be created with a tip having thermal energy or cryogenic energy. After the device 81 is appropriately implanted, the applicator 55 is withdrawn and the trabecular meshwork surgery is concluded.

The principles of the hydrogel coating can be applied to coat a microstent that is implantable within a body channel (for example, a cardiovascular stent, an esophagus stent or the like), the microstent comprising a tubular mesh having an outer circumferential periphery; and a swellable substrate incorporated about at least a portion of the outer circumferential periphery of the tubular mesh, wherein the substrate swellably expands radially outwardly after implantation that is adapted suitably for retention within the body channel. In one embodiment, the tubular mesh is retractably expandable radially.

Some aspects of the invention provide a method of implanting a swellable microstent within an eye comprising creating an incision through a conjunctival tissue at a limbus; radially incising an junction between an angle tissue and sclera, which is surgically extended until Schlemm's canal is entered posteriorly; and placing a swellable microstent between the anterior chamber and Schlemm's canal of the eye, wherein the microstent swells after implantation that is adapted suitably for stent retention within the eye. The microstent comprises swellable hydrogel or hydrolytically degradable hydrogel.

A preferred embodiment provides a method of implanting a swellable microstent within an eye comprising creating an incision through a cornea; incising an opening through trabecular meshwork, which is surgically extended until Schlemm's canal is entered anteriorly; and placing the swellable microstent between the anterior chamber and Schlemm's canal of the eye, wherein the microstent swells after implantation that is adapted suitably for stent retention within the eye. The microstent comprises swellable hydrogel and/or hydrolytically degradable hydrogel.

A further aspect of the invention provides a method of treating glaucoma, the method comprising providing at least one pharmaceutical substance incorporated into a trabecular microstent; implanting the microstent within a trabecular meshwork of an eye such that a first end of the microstent is positioned in the anterior chamber of the eye while a second end is positioned in a Schlemm's canal, wherein the first and second ends of the microstent establish a fluid communication between the anterior chamber and the Schlemm's canal; and allowing the microstent to release a quantity of the pharmaceutical substance into the eye.

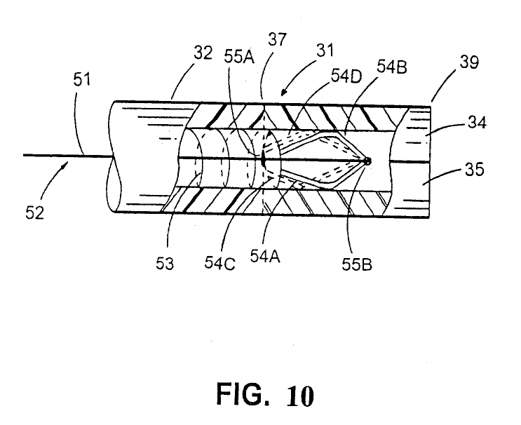

FIG. 10 shows another preferred embodiment of the trabecular stent constructed according to the principles of the disclosure. A delivery applicator 52 may be placed inside a lumen of the hollow elongate tubular element, wherein the delivery applicator comprises a deployment mechanism for effecting the outlet section to substantially expanded. The delivery applicator may be selected from a group consisting of a guidewire, an expandable basket, an inflatable balloon, or other expanding mechanism. In one embodiment, a delivery applicator 52 with an expandable basket comprises a plurality of expandable members 54A, 54B, 54C, 54D that all securely joined at a proximal joint 55A and at a distal joint point 55B. A distal end of a push-pull type wire 51 is also joined at the distal joint point 55B. The proximal joint 55A is located at the distal end of a compact guidewire 53 of the delivery applicator. Therefore, by pulling the push-pull wire 51 of the delivery applicator toward the operator, each of the expandable members 54A, 54B, 54C, 54D expand radially outwardly so as to effect the outward pushing action for the outlet section.

U.S. Pat. No. 6,077,298, U.S. patent application Ser. No. 09/596,781, filed Jun. 19, 2000, and U.S. patent application Ser. No. 09/847,523, filed May 2, 2001, the entire contents of which are incorporated herein by reference, disclose medical devices made of shape-memory Nitinol having a shape-transition temperature. In an embodiment, a trabecular stent comprises a hollow elongate tubular element having an inlet section and an outlet section, wherein the outlet section comprises an expandable element adapted to be positioned and stabilized inside Schlemm's canal. The expandable element may be made of a shape-memory material such as shape-memory Nitinol or shape-memory plastic material. In a preferred embodiment, the shape-memory Nitinol has a preshape and a shape-transition temperature, wherein the shape-memory Nitinol expands to its preshape when the shape-memory Nitinol is heated to above the shape-transition temperature.

The shape-transition temperature for the shape-memory Nitinol is preferably between about 39° C. and about 90° C. The shape-transition temperature is more preferred between about 39° C. and 45° C. so as to minimize tissue damage. An external heat source may be provided and adapted for heating the shape-memory Nitinol to above the shape-transition temperature of the shape-memory Nitinol. Examples of such external heat sources include a heating pad, a warm cloth, a bag of warm water, remotely deliverable heat, electromagnetic field, and the like. In another embodiment, the shape-memory Nitinol may be embedded within a biocompatible material selected from, for example, silicone, polyurethane, porous material, expanded polytetrafluoroethylene, semi-permeable membrane, elastomer, and mixture of the biocompatible material thereof. In general, the expandable element is relatively flexible and soft so that it does not impart undesired force or pressure onto the surrounding tissue during and after the deployment state.

In one embodiment, the trabecular stent of the present disclosure may have a length between about 0.3 mm to over a few millimeters. The outside diameter of the trabecular stent may range from about 30 μm to about 500 μm or more. The lumen diameter is preferably in the range of about 20 μm to about 150 μm or larger. The outlet section may be curved or angled.

In one embodiment, means for forming a hole/opening in the trabecular mesh 21 may comprise using a sharpened applicator or a screw shaped applicator.

In a preferred embodiment of the trabecular meshwork surgery, the patient is placed in the supine position, prepped, draped and anesthesia obtained. In one embodiment, a small (generally less than 1-mm) self-sealing incision is made. Through the cornea opposite the stent placement site, an incision is made in the trabecular meshwork with an irrigating knife. The stent is then advanced through the corneal incision across the anterior chamber held in a delivery apparatus or delivery applicator under gonioscopic (lens) or endoscopic guidance. The apparatus or applicator is withdrawn from the patient and the surgery is concluded. The delivery apparatus or applicator may be within a size range of 20 to 40 gauges, and preferably about 30 gauges. This is a typical ab interno procedure disclosed herein.

In a further embodiment, a method for increasing aqueous humor outflow in an eye of a patient to reduce intraocular pressure therein comprises: (a) creating an opening in trabecular meshwork by an applicator; (b) inserting a trabecular stent through the opening, wherein the trabecular stent comprises an inlet section and an outlet section, and wherein the outlet section comprises an expandable element adapted to be positioned and stabilized inside Schlemm's canal; and (c) expanding the expandable element to position inside Schlemm's canal.

Although exemplary embodiments of the invention have been described, certain variations and modifications will be apparent to those skilled in the art, including embodiments that do not provide all of the features and benefits described herein. Accordingly, the scope of the present invention is not to be limited by the illustrations or the foregoing description, but rather solely by reference to the claims and their equivalents.

What is claimed is:

1. An implant for use in an eye with glaucoma, said implant comprising:
an inlet section in fluid communication with an outlet section, said inlet section being sized and shaped to fit at least partially in the anterior chamber of said eye, and said outlet section being sized and shaped to fit at least partially in Schlemm's canal of said eye;
wherein said implant comprises a substrate that is expandable in the eye to assist in retaining the implant in the eye.

2. The implant of claim 1, wherein the substrate is at the inlet section.

3. The implant of claim 1, wherein the substrate is at the outlet section.

4. The implant of claim 1, wherein a coating on the inlet and/or outlet sections comprises the expandable substrate.

5. The implant of claim 1, wherein said implant comprises a material selected from the group consisting of titanium, stainless steel, silicone, polyurethane, polyvinyl alcohol, polyvinyl pyrolidone, collagen, heparinized collagen, polytetrafluoroethylene, expanded polytetrafluoroethylene, fluorinated polymer, fluorinated elastomer, flexible fused silica, polyolefin, polyester, and polysilicon.

6. The implant of claim 1, wherein said implant comprises a biodegradable material selected from the group consisting of poly(lactic acid), polyethylene-vinyl acetate, poly(lactic-co-glycolic acid), poly(D,L-lactide), poly(D,L-lactide-co-trimethylene carbonate), poly(caprolactone), and poly(glycolic acid).

7. The implant of claim 1, wherein the expandable substrate comprises a hydrogel.

8. The implant of claim 7, wherein the hydrogel is hydrolytically degradable.

9. The implant of claim 1, further comprising at least one therapeutic agent selected from the group consisting of heparin, beta-adrenergic antagonists, TGF-beta, anti-glaucoma drugs, and antibiotics.

10. The implant of claim 1, further comprising at least one therapeutic agent selected from the group consisting of a gene, a growth factor, and an enzyme.

11. The implant of claim 1, wherein said implant is substantially axisymmetric.

12. A surgical method treating glaucoma in an eye comprising:
incising through the sclera of the eye and into Schlemm's canal of the eye;
placing an implant, having an inlet section in fluid communication with an outlet section, through said scleral incision into the eye such that the inlet section of the implant resides at least partially in the anterior chamber of the eye, and the outlet section resides at least partially in Schlemm's canal of said eye; and
expanding a substrate on the implant to assist in retaining the implant in the eye.

13. The method of claim 12, wherein said substrate comprises a hydrogel.

14. A surgical method treating glaucoma in an eye comprising:
incising through the cornea of the eye;
placing an implant, having an inlet section in fluid communication with an outlet section, through said corneal incision into the anterior chamber of the eye;
positioning the implant such that the inlet section of the implant resides at least partially in the anterior chamber of the eye, and the outlet section resides at least partially in Schlemm's canal of said eye; and
expanding a substrate on the implant to assist in retaining the implant in the eye.

15. The method of claim 14, wherein said substrate comprises a hydrogel.
